(19) United States
(12) Patent Application Publication
(10) Pub. No.: US 2007/0275095 A1
(43) Pub. Date: Nov. 29, 2007

(54) TREATMENT OF INFLAMMATORY, AUTOIMMUNE, OR OTHER DISORDERS, USING AGENTS THAT REDUCE THE SEQUESTERING OF ZINC BY CALPROTECTIN

(76) Inventor: David C. Kossor, Ellisville, MO (US)

Correspondence Address: PATRICK D. KELLY, 11939 MANCHESTER #403, ST. LOUIS, MO, 63131, US

(21) Appl. No.: 11/627,746

(22) Filed: Jan. 26, 2007

(30) Foreign Application Priority Data
Jul. 26, 2005 (US) PCT/US05/26413

(51) Int. Cl. A61K 33/30; A61K 31/4045; A61K 38/00; A61M 37/00; A61P 19/02; A61P 29/00; A61P 25/28; A61P 1/16; A61K 38/45; A61K 31/65

(52) U.S. Cl. 424/643; 424/94.5; 514/152; 514/2; 514/415; 514/789; 604/6.01

(57) ABSTRACT

Treatments are disclosed for inflammatory, autoimmune, or other disorders characterized by excessive activity of calprotectin, a protein that normally defends against microbial infections by sequestering available zinc, at a site of infection. Excessive calprotectin activity, which can cause zinc deficiencies in localized tissues, can create or aggravate various disorders. However, ingestion of systemic (oral) zinc supplements tends to activate offsetting mechanisms, and such supplements therefore usually are ineffective. Accordingly, targeted treatments are disclosed herein for suppressing and controlling excessive calprotectin activity, in local tissues. Such methods include targeted injections of zinc solutions, and plasmapheresis treatment. Screening tests also are described for identifying non-protein drugs that can either (i) bind specifically to the zinc-binding sites of calprotectin, or (ii) suppress the release of calprotectin by neutrophil cells.

### RELATED APPLICATION

This application claims the benefit of PCT application number PCT/US2005/026413, filed on Jul. 26, 2005 and published as WO/2006/014911.

### BACKGROUND

The invention relates to biochemistry and medicine, and to a protein called calprotectin, which binds to calcium and zinc in body fluids. It has been used in the past as a marker and diagnostic indicator for certain diseases. This invention discloses therapeutic interventions that can control calprotectin activity in various diseases, such as some autoimmune diseases.

Calprotectin is one of several names given to a certain protein that, under normal conditions, helps humans or other mammals fight bacterial infections. Because this protein became of interest to a number of research teams that approached it from different angles, and because it is made up of two polypeptide subunits that belong to a known family of polypeptides, calprotectin and its subunits have been given a number of different names, which require a brief listing and summary.

First, it should be noted that calprotectin is formed from two different subunits that bind to each other. That binding reaction is not covalent; instead, it is comparable to an antibody binding to an antigen. Since the two subunits are different from each other, calprotectin is considered and described as a “hetero-dimer”. There also is evidence that some calprotectin contains two copies of the light subunit and one copy of the heavy subunit, and still other calprotectin apparently contains two copies of each subunit.

Both of the two subunits and their genes were fully sequenced by the late 1980's (Odink et al 1987; Lagasse et al 1988; Andersson et al 1988), and their crystalline structure has been determined (Itou et al 2001 and 2002; also see Moncrief et al 1990, Raftery et al 1996 and 1999, Loomans et al 1998, and Rety 2000 for additional information on the folding, conformation, and structure of the subunits). Both subunits belong to a class of polypeptides called S100 peptides. The lighter subunit, which has 93 amino acid residues and a molecular weight of about 11 kilodaltons (kDa), usually is called S100A8, but it is also called the MRP8 protein (MRP is derived from the phrase “migration inhibitory factor related protein”), the L1L protein (derived from leukocyte-derived light chain), or calgranulin A. The heavier subunit, which has 114 amino acid residues and a molecular weight of about 14 kilodaltons (kDa), usually is called S100A9, but it is also called the MRP 14 peptide, the L1H peptide, or calgranulin B.

The complete protein, formed when two or more subunits bind to each other, is called calprotectin, the S100A8/S100A9 protein, the MRP8/14 protein, or the 27E10 antigen. It is also sometimes called “the cystic fibrosis antigen”; however, the S100A8 (MRP8) and the S100A9 (MRP14) subunits also apparently have been referred to in various articles as the cystic fibrosis antigen.

Calprotectin plays an important role in a mammalian system that is usually called the innate immune system. That term can be understood by considering how and why it is different from the adaptive immune system.

The adaptive immune system requires and uses antibodies, which are made and used with the involvement of various white blood cell types, including B cells, T cells, macrophages, killer cells, etc. Antibodies are proteins with variable sequences, created by complex genetic rearrangements within the chromosomes of certain types of white blood cells. A wide assortment of antibodies is effectively thrown up against a population of invading bacteria, viruses, or other microbes, and some of those antibodies will bind to proteins on the surfaces of the microbes. Those antibodies that bind to the invading microbes will be identified, selected, and then mass-produced, by means of still more complex processes.

The process of generating an assortment of antibodies, selecting specific antibodies that have managed to bind to a specific type of invading microbe, and mass-producing the antibodies that happen to be effective in combatting a particular infection, usually takes several days. That delay can allow most types of bacteria and viruses (which can reproduce many times faster than mammalian cells) to generate huge numbers of invading microbes, before a complete defensive response that requires antibodies can move fully into action. That delay, before the adaptive immune system can mount an effective full-scale defense, is why vaccines (if prepared and administered in ways that allow the immune system to get ready, in advance, for an infection), can make a huge difference in how severe a disease or infection will become.

That delay of several days, before the adaptive immune system can fully respond, also explains why mammals evolved with roughly half a dozen types of specialized proteins and cell types that can respond almost immediately, to help the body fight off and slow down a set of invading microbes. This type of “first-line” response will help slow down the initial assault, while reinforcements and heavy artillery (i.e., the adaptive immune system, with antibodies, B cells, T cells, etc.) are being prepared and move into position. The “first line” defensive cells and molecules must be able to respond and act almost immediately, without having to wait several days for an antibody response to be prepared.

Accordingly, the defensive molecules that can respond immediately to invading microbes, and the cells that carry them around and release them at sites of infection, are called the “innate” immune system. This system is also regarded by some as a “primitive” immune system, and it is still the main line of defense among some types of insects, worms, and other lower animals that do not have immune systems with antibodies.

The innate immune system in humans and other mammals includes calprotectin, which is carried in very high concentrations by white blood cells called “neutrophils”. Calprotectin makes up an estimated 45% to 60% of all the water-soluble proteins carried by neutrophils. Its antimicrobial activities are discussed in articles such as Steinbakk et al 1990, and Sohnle et al 1991, 2000a, and 2000b.

When neutrophil cells are attracted to the site of an infection, they release their calprotectin. The calprotectin then grabs and “sequesters” any available zinc (i.e., it binds tightly to any available zinc ions, in a way that renders the zinc unavailable to the invading microbes). An understanding of the importance of this process requires some background information on the roles and actions of zinc, in mammalian physiology.

Zinc in Bodily Fluids and Tissues

In body fluids, under physiological conditions, zinc exists in either “free” form (which usually is in the form of positively charged ions, Zn⁺⁺), or in “bound” forms in which it is associated with proteins or other molecules. “Free” zinc can also be referred to by terms such as unbound, exchangeable, rapidly exchangeable, available, accessible, chelatable, bindable, labile, etc. It also needs to be recognized that even in “bound” forms of zinc, the strength of the binding can range from relatively loose, to very tight. As a result, free and bound zinc will generally establish a dynamic and adaptive equilibrium that can change in various fluid or cell types, and over time.

Zinc ions play crucial roles in stabilizing the three-dimensional shapes of many enzymes and other proteins; a single ion of zinc can form up to four stable cross-linking bonds with cysteine and histidine residues in proteins. This role of zinc, in stabilizing proteins, evolved over the eons because zinc is a benign transition metal; unlike iron, copper, or other metals, it poses no reduction or oxidation threat to proteins or DNA (in fact, zinc can interrupt oxidation and reduction processes and cycles caused by other agents; this renders it useful as a protective and beneficial antioxidant).

In addition, zinc also helps create the catalytic activity that is essential for many enzymes. In general, it does this by participating in various types of electron transfers, which are essential for breaking old bonds and forming new bonds.

As a result of these properties and activities, zinc became essential to literally thousands of enzymes and structural proteins, and it is essential to the growth and reproduction of most types of microbes.

More information on how zinc binds in an equilibrium-seeking manner to various amino acids, proteins, and other biological molecules, with varying degrees of strength and tightness (also referred to as affinity, avidity, or other terms), is available in books such as Frederickson et al 1984, Mills et al 1989, and Prasad 1994, and in review articles such as Vallee et al 1993, Berg et al 1996, and Frederickson et al 2005.

Accordingly, available zinc is enormously important to all cells, including both (i) invading microbes, and (ii) the cells of an animal host. Therefore, when calprotectin that has been released by neutrophil cells of a mammal, as a rapid defensive mechanism, binds tightly to and sequesters any available zinc in a localized area or segment of tissue, the available zinc cannot be used by invading microbes. This prevents the microbes from being able to obtain enough zinc to grow and reproduce at uncontrolled rates. This “first response” defense helps keep an infection under control, while a larger antibody response is being prepared. This is described in more detail in articles such as Striz et al 2004, a review article with numerous other articles cited therein.

Although the “innate immune” functions of calprotectin are essential for helping mammals defend against invading microbes, researchers have recognized in recent years that some types and cases of inflammatory conditions (such as rheumatoid arthritis, multiple sclerosis, inflammatory dermatoses, cystic fibrosis, and certain other disorders) are characterized by excessive amounts of calprotectin. Consequently, high levels of calprotectin in certain types of disorders are being used and/or evaluated as a marker, indicator, or diagnostic tool, to help physicians and patients monitor the severity, status, activity levels, or other aspects of various disorders.

As one example, at least half a dozen major types of long-term intestinal and/or bowel disorders are known, including Crohn's disease, ulcerative colitis, irritable bowel syndrome, colon cancer, etc. It has been found that some but not all of those disorders (including Crohn's disease and ulcerative colitis, as described in U.S. Pat. No. 5,455,160 (Fagerhol et al 1995) and Tibble et al 2000) lead to elevated concentrations of calprotectin in fecal matter (i.e., excrement). As a result, levels of calprotectin in feces can be used to distinguish Crohn's disease or ulcerative colitis from other bowel disorders, and to assess the severity of the disorder as it goes through cycles of flareups and remissions. Increased concentrations of calprotectin also are found as a side effect in some types of cancer, including colorectal carcinoma, squamous carcinomas of the lung and bladder, and some types of lymphomas.

Although increased concentrations of calprotectin are being used as diagnostic agents (or markers, biomarkers, etc.) to assess the severity of various inflammatory diseases, cancers, etc., the consequences of the increased local sequestration of zinc by calprotectin has not been evaluated, and it is believed by the Inventor herein that in some cases, in some patients, conditions are being created where calprotectin responses and activities reach excessive levels that begin to starve local areas or types of tissues of zinc.

This belief is consistent with, and supported by, various factors that are known about the roles of zinc in wound repair and healing mechanisms that are active in skin and other connective tissues. For example, Savlov et al 1962 reported that when zinc was fed to rats that were then subjected to skin incisions, the zinc was deposited in the active healing area during the time that the epidermal cells around the incision were multiplying rapidly; however, the zinc did not persist in significant amounts in the scar tissue that remained at the wound site. Henzel et al 1970 reported that in animals with skin incisions, zinc was sequestered in relatively high quantities in the wound area during active healing, even in test animals that had been subjected to zinc-deficient diets to reduce their total stores of zinc. Agren 1990 reported that in lab animals, topically-applied zinc accelerated the growth of epidermal cells and the closure and healing of experimentally-inflicted cuts, and Other reports which indicate that zinc can help accelerate the healing of skin wounds, skin ulcers in diabetics, etc., include Pories et al 1967, Husain 1969, and Hallbook et al 1972, Stromberg et al 1984, and Apelqvist et al 1990. In addition, on a practical level, zinc has been a primary active ingredient in ointments for treating diaper rash, for decades.

In addition, zinc is an essential cofactor in enzymes called metalloproteinases, which play crucial roles in manipulating and “remodeling” collagen, the fibrous protein that holds together cells in connective tissues, in animals. Collagen fibers that are more than a few weeks old are constantly being degraded, so they can be replaced by new collagen, which is gradually secreted by various types of cells. This is a major process in all higher animals; in humans, it enables muscles and connective tissue to remain strong and flexible over a span of numerous decades. The degradation and removal of old collagen, and its replacement by new collagen, require direct and continuing involvement by metalloproteinases, and those enzymes simply cannot function without zinc. Furthermore, metalloproteinase defects or alterations are directly involved in a number of diseases, including angiogenesis and the growth of cancerous tumors, lung diseases, and myocardial problems. The roles of metalloproteinase enzymes in connective tissues, and in various diseases, are described in articles such as Woessner 1991, Isakson et al 2001, and Pardo et al 2005. Since zinc is essential for metalloproteinase enzyme activity, localized zinc deficiencies can pose risks of disrupting the maintenance and gradual turnover of collagen fibers, in connective tissues.

However, despite all of the foregoing factors, the types of diseases and problems listed above cannot be solved, in simple and straightforward ways, merely by feeding or otherwise administering more zinc to an animal. The reason for this is that cells require and maintain an “optimum” concentration of zinc, which creates an effect that is analogous to a “double-edge sword”. Although too little zinc creates problems, too much zinc also can be equally damaging. If large quantities of zinc are administered to the entire body, in an indiscriminate manner (such as by oral ingestion), the body will soon detect that a potentially dangerous oversupply condition has been reached or is being approached, and it will respond by taking steps to control and reduce that oversupply. Those types of natural responsive mechanisms often lead to other, sometimes unexpected and occasionally offsetting actions, by systems that are attempting to sustain the equilibrium-seeking “homeostasis” nature of zinc in a mammalian body (homeostasis is a medical term that refers to a dynamic and adaptive equilibrium; an animal body attempts to maintain its homeostasis, despite fluctuations in food intake, outside factors, etc.).

As a classic example, physicians and researchers have tried for years to treat cases of Crohn's disease, by administering relatively large quantities of zinc (such as oral dosages of 50 mg/day, or even higher) to patients who suffer from the disease. However, instead of providing substantial and lasting benefits for such patients, that type of “non-targeted” approach to treatment (using system-wide administration of zinc pills that are ingested orally) typically induces the formation of certain types of “storage or elimination” proteins, notably including various isoforms of metallothionein. Metallothionein will transport zinc (in bound form) to the liver, which will then mix the zinc with bile, leading to excretion of elevated quantities of zinc, in feces. Indeed, the triggering of high levels of metallothionein expression (which is directly inducible, by high concentrations of zinc) can even lead to relative deficits in zinc, in response to oral ingestion of zinc at relatively high levels. That is a paradoxical response, but it must be understood and appreciated, to recognize why simple zinc supplementation has not been able to effectively treat various disorders and diseases that are known to involve calprotectin over-expression and/or localized zinc deficiencies. That and similar problems are illustrated in articles such as Sampson et al 2002 and Saito et al 2002.

To the best of the Applicant's knowledge and belief, prior researchers have not taught or suggested that elevated calprotectin levels can actually create or aggravate additional medical problems, or that taking steps to intervene in and modulate the calprotectin system, in ways that control and reduce calprotectin activity in stressed tissues, may be able to help reduce and control various medical problems that are not merely associated with elevated calprotectin, but that may be aggravated by elevated calprotectin.

Beyond that, based on extensive readings in the literature, it appears that essentially all researchers who are working in this field tend to presume and believe that:

(1) the essential and important effects of calprotectin, when released by neutrophils, is to suppress microbial growth;

(2) because of the numerous adaptive and balanced processes, mechanisms, and molecules that are involved in regulating zinc concentrations and zinc homeostasis in bodily fluids, the tissues involved will be able to tolerate any temporary and transitory localized decreases in zinc that may be caused by calprotectin release, and can reestablish healthy and appropriate levels of zinc in ways that will avoid and minimize any tissue injury.

Based on his readings, research, and insights, the Inventor/Applicant herein has become convinced of the opposite, and believes that in at least some and possibly many cases of ongoing inflammatory diseases and certain other diseases, chronic and sustained release of inappropriately high quantities of calprotectin molecules, by neutrophils that continue responding over a span of months or years to such inflammatory or other conditions, apparently becomes a major and crucially important process, in the ongoing stress and damage that is being inflicted on the stressed and damaged tissues that are involved.

Before the objects of the invention are summarized, and before the actual steps and mechanisms of the treatments herein are described, several general medical terms need to be defined.

Any references herein to terms such as therapy, treatments, etc., are limited to interventions that reduce calprotectin levels, and that help relieve suffering or discomfort and improve the health, quality of life, or similar conditions of a patient in need of such treatment, by reducing the severity, retarding the progression, or alleviating the symptoms of a disease or disorder. It has been known for years that calprotectin concentrations in feces or body fluids can be used in diagnostic methods for measuring and monitoring various diseases. However, this current invention does not relate to diagnosis, measuring, or monitoring of diseases; instead, it is limited to therapy and treatment of diseases.

For convenience, the term “disease” as used herein includes conditions that are often referred to as disorders, syndromes, or similar terms by medical professionals. This excludes traumas and other external injuries, and it excludes microbial infections. However, diseases can and often do arise from bodily repair processes that have gone awry in ways that create or aggravate a chronic, degenerative, or similar problem when the body attempts to recover from a trauma or infection.

As in common usage, “disease” implies that a medical problem has a level of severity and importance that merits medical attention; however, this does not require that a person must actually seek medical attention, since such decisions often depend on factors such as cost, accessibility, insurance coverage, etc. Instead, the phrase is intended to indicate that a level of discomfort has risen above the level of a typical headache, muscle ache or soreness, or other minor annoyance that accompanies aging, and has reached a point (which can be cause by short-term severity, or by a chronic and lingering condition that does not resolve and disappear over a reasonable time) where medical attention by a skilled professional would be helpful and well-advised, if the person can afford it.

The term “disorder” as used herein refers to an underlying biochemical problem that causes or aggravates a disease as defined above. In the discussion herein, these disorders involve (usually as a triggering or aggravating factor, or as a link in a chain or cascade) a calprotectin concentration that is chronically elevated, in a manner that leads to localized deficits in available zinc concentrations.

For convenience, the term “patient” refers to a person who is suffering from a calprotectin-related disease or disorder as described herein, or who can substantially benefit from a treatment as disclosed herein, regardless of whether such person actually seeks medical attention or treatment.

“Medical attention” is not limited to treatment by licensed physicians; instead, it refers to a treatment that can address a problem that can and should properly be regarded as a medical problem. In the real world, people who suffer from various minor aches and pains (and friends, family, neighbors, pharmacists, nurses and other caregivers, etc.) often can recognize the symptoms of a problem, and can use or recommend helpful yet relatively low-cost treatments (such as, for example, over-the-counter treatments that can be purchased in drugstores) without requiring a visit to a physician.

References to “local” (or localized, etc.) zinc deficiencies are not intended in a strict manner, and instead can include nearly any type or level of zinc deficiency that is not system-wide. As one example, it is well known that cystic fibrosis directly affects the lungs; it is less well known that it also affects the liver. Accordingly, calprotectin over-expression and zinc deficiencies in cystic fibrosis patients may be affecting both the liver and the lungs, in various patients. Such effects are regarded as localized, as that term is used herein.

Similarly, rheumatoid arthritis (as with any other form of arthritis) tends to attack articulating joints with cartilage segments. Accordingly, a problem that arises in multiple body parts that share certain traits (such as articulating joints, which contain cartilage segments), would be regarded herein as a problem in local tissues, rather than a systemic problem, even if the “common trait” tissues occur in various distributed tissues located in different parts of the body.

Two other terms need to be defined. Calprotectin molecules that have zinc binding sites that are un-occupied, and that are therefore ready to receive and chelate zinc ions, are referred to as active calprotectin. By contrast, calprotectin molecules with zinc binding sites that are already occupied (or saturated, etc.), either by zinc or by “calprotectin suppressing drugs” (CSD's), as described below, are referred to as inactive (or inactivated) calprotectin, since they no longer have the ability to bind to zinc ions.

Accordingly, one object of this invention is to disclose methods and agents for therapeutic treatment (as distinct from diagnosis or monitoring) of inflammatory or other disorders that are caused, aggravated, or otherwise related to or characterized by elevated concentrations of calprotectin.

Another object of this invention is to disclose that “targeted” methods for reducing concentrations or activity levels of calprotectin in specific localized tissues can provide effective ways for treating various disorders inflammatory or other disorders that are caused, aggravated, or otherwise related to or characterized by elevated concentrations of calprotectin.

Another object of this invention is to disclose that once the “targeted” methods for reducing concentrations or activity levels of calprotectin in specific localized tissues have been shown to be effective, in “proof of principle” tests, screening methods can be used to identify nonprotein small-molecule “calprotectin-suppressing drugs” (CSD's) that will either: (i) bind to calprotectin in ways that will block, reduce, or otherwise modulate the sequestering of zinc by excess calprotectin, or (ii) help suppress the release of calprotectin molecules, by neutrophil cells. Either of those two approaches can provide therapeutic benefits for at least some inflammatory or other disorders that are aggravated by elevated concentrations of calprotectin.

These and other objects of the invention will become more apparent through the following summary, drawings, and detailed description.

### SUMMARY OF THE INVENTION

Therapeutic treatments are disclosed for various types of inflammatory, autoimmune, or other disorders that are aggravated or otherwise characterized by excessive concentrations of calprotectin, a protein normally carried by leukocytes. In healthy people, calprotectin is released by leukocytes at the site of a microbial infection. It provide a first line of defense against microbial growth, by sequestering any available zinc. Since zinc is essential for microbial growth and reproduction, this initial response helps control and limit an infection, while a complete antibody response is being generated.

It is disclosed herein that derangements of the calprotectin defense mechanism hold an important key to at least some types and/or cases of autoimmune and/or inflammatory disorders, arising from local or regional deficiencies of zinc. Among other factors, zinc is essential to cell reproduction, tissue growth, and numerous enzyme activities, and it plays crucial roles in the repair and healing of damaged tissues. Therefore, local or regional deficiencies of zinc, caused by excessive activity of calprotectin, can seriously hinder the natural repair mechanisms that healthy tissues use to keep inflammatory outbreaks under proper regulation and control.

The appreciation for the ability of calprotectin to induce a localized or regional zinc deficiency, in ways that can be treated effectively and therapeutically when approached in a properly targeted manner, is a novel concept that previously has gone unrecognized. Prior tests and studies focused only on systemic indicators of zinc deficiency, such as total concentrations of both free and bound zinc in circulating blood (such concentrations are often tested by measuring the activity levels of various zinc-dependent enzymes, such as carbonic anhydrase, alkaline phosphatases, etc.). As a result, various attempts to use orally-ingested (systemic) zinc supplements, to treat various diseases (such as Crohn's disease, as just one example), have failed to provide effective treatments. Because of apparently paradoxical factors that arise from the body's multiple mechanisms for attempting to sustain zinc equilibrium and homeostasis, efforts to administer oral (systemic) zinc supplements for diseases that are known to involve elevated levels of calprotectin either had no significant effect, or they apparently “tricked” the body into thinking it had too much zinc, thereby triggering responsive and compensatory processes, which caused the body to begin acting as though it had too much zinc, leading to more problems rather than to therapeutic benefits. In addition, researchers have been reluctant to make any serious attempts to use drugs that cause system-wide suppression of the entire calprotectin system, since they could be impairing a crucially important part of the “innate” immune system, which provides a rapid “first line” defense against microbial invasions while the “adaptive” immune system takes longer to prepare a complete antibody response.

Accordingly, two different approaches are disclosed herein, which can use currently available devices, methods, and reagents to provide targeted forms of therapy that will aim and focus their effects on specific targeted organs, limbs, or tissue types that are indeed suffering from localized zinc deficiencies, which are being caused by chronic calprotectin surpluses. One approach involves arterial infusion of a liquid that contains a zinc buffer, directly into an artery that supplies affected tissues. The liquid will provide sufficient quantities of zinc (in a suitable transport or buffer compound) to saturate any active calprotectin molecules in the region, thereby converting them into zinc-saturated and therefore inactive calprotectin molecules. The liquid will then provide additional zinc to the tissues that previously were struggling with zinc deficits caused by excess levels of active calprotectin.

The other approach involves “extra-corporeal” blood processing (also called “plasmapheresis”). In this approach, which is comparable in some respects to dialysis, blood is withdrawn from a vein that directly services affected tissues. The blood is centrifuged to remove the cells, and the plasma or serum is passed through a device (such as an affinity column containing monoclonal antibodies) that removes calprotectin from the plasma or serum. The “scrubbed” plasma or serum emerges from the device and is remixed with the cells, and the “scrubbed” blood (with its calprotectin load removed) is returned to the patient.

Either or both of these approaches can provide therapeutic benefits, using currently available technology; and, for some patients with certain disorders (such as inflammatory conditions that may completely resolve if the affected tissues are given a chance to heal), reasonably short periods of treatment may be able to provide lasting benefits and possibly even permanent cures.

However, as with most cases of long-term dialysis, the foregoing approaches do not provide ideal solutions. Therefore, once these approaches have shown “proof of concept” results, pharmaceutical companies will be encouraged to screen their “libraries” of candidate drug compounds, to identify molecules that can either: (i) bind strongly and competitively to the zinc binding sites of calprotectin, thereby occupying and inactivating those sites; or, (ii) suppress the release of calprotectin molecules, by neutrophil cells that carry calprotectin. Molecules that can perform these tasks are referred to herein as “calprotectin suppressing drugs” (CSD's).

Several compounds that can provide “starting point” or “baseline” compounds having some level of calprotectin suppressing activity are identified herein. Analogs, derivatives, and other variants of those known molecules can be created and screened for activity in suppressing calprotectin release or zinc sequestration.

### DETAILED DESCRIPTION

As summarized above, therapeutic treatments are disclosed for inflammatory, autoimmune, or other disorders that are caused or aggravated by excessive concentrations of calprotectin that create localized or regional zinc deficiencies.

It is not asserted or believed that the treatments disclosed herein will be able to treat and alleviate any and all inflammatory, autoimmune, or other diseases. Instead, it is asserted that such treatments will be able to provide, in a substantial, measurable, and statistically significant way, benefits for at least some patients who are suffering from at least some of the types of disorders mentioned herein. As the teachings herein are evaluated and tested in both animal models and human volunteers, patterns and percentages of efficacy in various population groups suffering from various disorders involving calprotectin dysfunction will become apparent. That information can then be taken into account by patients and physicians, in determining whether any such treatment should be used for a specific individual patient suffering from a specific disorder.

There are seven types or clusters of diseases that merit early evaluation, to assess the extent to which the treatments disclosed herein can help alleviate the damage and discomfort caused by such diseases. These clusters of diseases are briefly summarized, below. These brief descriptions are not offered or intended as authoritative analyses, and more extensive information on each cluster or type of disorder is available in numerous medical textbooks and review articles.

It also must be recognized that some or even most of these disease types or clusters have various overlaps. Indeed, because the calprotectin system and its various components and processes are an important part of the “innate” immune system, and because of the numerous correlations between inflammation and immune responses, essentially all of the diseases discussed herein that merit evaluation to determine whether and to what extent they will respond positively to treatment as disclosed herein will have at least some component or aspect that can be regarded as an inflammatory and/or autoimmune component. Nevertheless, in the discussion below, it should be noted that certain types of disease (including lupus, and rheumatoid arthritis, as examples) are regarded and referred to primarily as autoimmune diseases, rather than as diseases that have an autoimmune component.

With that as preface, the main diseases that merit early evaluation, using drugs that can modulate excess calprotectin expression, concentration, or activity in ways that will prevent or minimize localized or regional zinc deficits, can be grouped into the following clusters:

1. Rheumatoid arthritis;

2. Cystic fibrosis;

3. Inflammatory dermatoses that are characterized by surplus calprotectin levels, including psoriasis and some types of atopic dermatitis;

4. Inflammatory bowel diseases that are characterized by surplus calprotectin levels and/or apparent or inducible increases in nitrous or nitric oxides (collectively referred to as NO's), including ulcerative colitis and Crohn's disease;

5. Cancers that are characterized by elevated calprotectin levels, which includes some cases of colon or other gastrointestinal cancers, some liver and hepatobiliary cancers, some carcinomas of the head or neck, some cancers of the oral cavity, breast, pancreas, lungs, or ovaries, and some types of lymphomas and leukemias;

6. Liver diseases that are characterized by surplus calprotectin levels, including primary biliary cirrhosis (an autoimmune liver disorder that damages the septal and intrahepatic bile ducts, leading to fibrosis and other problems) and primary sclerosing cholangitis; and,

7. Various other diseases that involve apparent or inducible increases in nitrous or nitric oxides (collectively referred to as NO's, or occasionally as NOx or NOX where X is a variable that indicates that the compound(s) of interest might include any or all of NO, NO₂, NO₃, and possibly even N₂O) in localized tissues, or that are characterized by elevated levels of an enzyme called inducible nitric oxide synthase (iNOS); in particular, disorders that involve NO are of interest, because NO has an unstable and reactive “resonant” structure that is known to create or aggravate various inflammatory conditions.

In addition to those diseases, the treatments disclosed herein merit evaluation for their potential ability to help prevent or reduce various neurological diseases, potentially including Alzheimer's disease, dementia, multiple sclerosis, and autism. It is worth noting, in particular, that some case reports have suggested that the apparent onset of autism, in some small children, was preceded by a severe bout of gastrointestinal distress. This raises questions as to whether such episodes may have triggered transitory calprotectin over-expression levels, and transitory zinc deficits, that may have inflicted lasting and even permanent damage on neonates or infants because they occurred during critical growth stages.

A comprehensive analysis of the diseases listed above, which would explain why at least some occurrences of each of these diseases are believed to be correlated with elevated calprotectin levels and local or regional zinc deficiencies, is beyond the scope of this patent application. Those who wish to locate information that correlates the diseases listed above with elevated calprotectin levels and/or local or regional zinc deficiencies can locate such information by means of database searches, beginning with the free database operated by the National Library of Medicine.

It is believed that derangements of the calprotectin defense mechanism, and resulting depressions in locally available zinc supplies, hold important keys to at least some cases of nearly all of the diseases listed above. Among other factors, zinc is essential to cell reproduction and tissue growth, and it plays a crucial role both in the repair and healing of damaged tissues, and in the normal and gradual replacement of aging collagen fibers by new collagen fibers, in connective tissues. Therefore, as a general principle, it is believed that local or regional deficiencies of zinc, if caused by excessive activity of calprotectin, can hinder the natural repair and replacement mechanisms that healthy tissues use to keep inflammatory outbreaks under proper regulation and control, and to carry out the normal and gradual replacement of aged collagen fibers with new collagen fibers.

Accordingly, agents that can competitively bind to calprotectin, preferably at its zinc binding sites, can help reduce the tendency of excessively high calprotectin levels, in inflamed or otherwise stressed tissues, to create or aggravate local or regional zinc deficiencies that cause or aggravate inflammatory, autoimmune, or other disease processes.

As this is being written, the two best treatments that can be provided for reducing excessive calprotectin activity in localized tissues, using already-known methods, devices, and reagents, are described in the two sections below. Both of these methods can be used in conjunction with each other, if desired.

However, it should be recognized from the outset that both of these two treatments fall short of being ideal. In at least some cases, instead of providing “cures” for the patients they will be used to treat, they will be comparable to requiring a lifelong regimen of insulin injections for diabetic patients, or dialysis sessions for kidney patients. Therefore, once these approaches have shown “proof of concept” results, pharmaceutical companies will be encouraged to screen their “libraries” of candidate drug compounds, to identify molecules that can either: (i) bind strongly and competitively to the zinc binding sites of calprotectin, thereby occupying and inactivating those sites; or, (ii) suppress the release of calprotectin molecules, by neutrophil cells that carry calprotectin. That screening approach is discussed in more detail below.

Targeted Injections or Infusions

The first method comprises targeted injections of a blood-compatible aqueous liquid (such as Ringer's lactate, a solution of glucose in buffered saline, etc.) that contains high concentrations of zinc, carried by a suitable transport or “buffer” compound, directly into an artery that supplies blood to localized tissues that are suffering from a calprotectin surplus and/or zinc deficit.

“Artery-specific” injections were initially developed for chemotherapy of solid tumors in specific organs (such as liver cancer), since they can allow physicians and oncologists to infuse a relatively toxic chemotherapeutic agent directly into a cancerous organ or tumor, at dosages that could not be tolerated at comparable levels if administered to the entire body. One such early system is described in U.S. Pat. No. 4,192,302 (Boddie 1980), which described artery-specific infusion of toxic chemotherapy agents into the liver, to treat liver cancer.

In the current invention, if desired, blood from one or more veins that carry blood out of the targeted localized tissue also can be chemically processed, to remove any unused or surplus zinc from the blood (or diluted blood) that emerges from the tissue being treated.

If this approach is used, the surplus free zinc in the injected blood mixture or other liquid will bind to the zinc-binding sites of the surplus calprotectin in the affected tissue, to a point that will saturate the zinc-binding sites of the calprotectin. This will prevent the calprotectin molecules from chelating and effectively withdrawing additional free zinc from the blood, lymph, or other liquids that surround and bathe the affected tissues. In addition, any remaining zinc that was supplied by the injected liquid can directly benefit the tissue that previously had been deprived of sufficient zinc, due to calprotectin's chelating activities.

Rather than carrying out this method of treatment using a single brief and relatively large “bolus” injection, a preferred method can be performed by using a slower infusion, over a span of, for example, one to several hours, in a manner comparable to a dialysis treatment. It also may be possible to develop and use “shunt” devices, analogous to shunts used in dialysis, to enable repeated infusions of a zinc-supplemented liquid into a particular targeted artery, over a span of multiple weeks or months. This type of approach also likely can be adapted to at-home and/or mobile treatments, which have become available for dialysis patients through the development of dialysis machines that are roughly the size of large briefcases, and that can be operated at home.

In some cases, and in some diseases, targeted zinc infusions may be able to help damaged tissue regain a stable improved “homeostatic set-point”, which may effectively comprise a state of health, mid-term or long-term remission, etc. In some cases, this may be able to completely eliminate the need for subsequent infusions, after localized damaged tissue has been effectively repaired. In other cases, a relatively intense round of frequent, high-dosage, or other “restorative” treatments may be able do sufficient good to allow any subsequent “maintenance” infusions to be reduced to a relatively infrequent basis, such as once a week, once a month, etc.

Candidate compounds that offer good candidates for evaluation as transport or buffer compounds, as described above, include complexes formed by reacting zinc with relatively weak organic acids, to form zinc gluconate, citrate, picolinate, etc.

Plasmapheresis (Extra-corporeal Blood Processing)

Another method of treatment that can be carried out using already-known and available methods, machines, and reagents comprises a process that is usually called plasmapheresis, or extra-corporeal blood processing. Briefly, it involves the following steps:

(1) removing a quantity of blood from the body of a patient, via a hollow needle inserted into a vein that carries blood from a localized area that needs treatment;

(2) using a centrifuge or other device to remove red and possibly white blood cells from the blood, since the cells would interfere with processing inside an affinity column;

(3) passing the remaining plasma or serum through a processing device that will remove calprotectin from the liquid that passes through the device. For example, in a preferred embodiment, the processing device can be an affinity column that has been loaded with a monoclonal antibody preparation. The monoclonal antibodies will bind to calprotectin, and they will be chemically affixed to beads that are trapped inside the column by filter screens at the inlet and outlet of the column;

(4) collecting the “scrubbed” plasma or serum that emerges from the affinity column or other processing device, and remixing the “scrubbed” plasma or serum with the blood cells that were removed in the earlier processing step, thereby reconstituting the patient's blood with a calprotectin load that has been greatly reduced by the out-of-body treatment; and,

(5) returning the “scrubbed” blood to the patient's body at a suitable location (if the blood has been passed through an oxygenator, the return site can be into an artery that is upstream from the tissue that is suffering from excess calprotectin levels).

In this manner, plasmapheresis (i.e., extra-corporeal blood processing) can remove calprotectin-loaded blood from a vein, pass it through a monoclonal antibody column or other device that will remove calprotectin from the blood, and then return the calprotectin-free blood to the patient's body.

Alternately or additionally, extra-corporeal blood processing can be used to reduce the numbers of neutrophil cells that are being sent to a localized tissue area that is suffering from a calprotectin surplus. This type of processing can use, for example, monoclonal antibodies that bind to certain antigens that are known to exist in abnormally large numbers on the surfaces of neutrophil cells. Such antigens are well-known, and are referred to as “human neutrophil antigens” (HNA's), as reviewed in Stroncek 2004. Alternately, methods for isolating neutrophils are known that use immobilized fragments or molecules that function as “chemotactic factors”, obtained from bacterial membranes, to attract neutrophil cells, as described in Russo et al 2003 and Vandal et al 2003.

In addition, anyone interested in this process should also become familiar with how platelets (also called megakaryocytes) are isolated from blood donors. Platelets are specialized types of blood cells, and are heavily involved in blood clotting. To treat people who suffer from hemophilia and other blood clotting disorders, platelets are isolated from blood provided by blood donors, and are made available for transfusions into hemophiliacs and others. The methods that are used to isolate platelets may be adaptable for removing neutrophils from the blood of people who are suffering from diseases that involve excessive calprotectin activity and local zinc deficits.

Either or both of the two methods summarized above (i.e., plasmapheresis, and targeted zinc infusions) can be used: (i) to directly confirm that calprotectin-reducing treatments can substantially benefit patients who are suffering from various diseases, such as the diseases listed above; and, (ii) to identify particular diseases and patient subpopulations that demonstrate the greatest benefits and improvements from such treatments.

While those processes and trials are being carried out and evaluated on the various classes of diseases mentioned above, researchers also can commence the process of identifying and testing candidate drug compounds that can be safely administered to patients suffering from calprotectin-related disorders, or to lab animals that provide models of such disorders. That process of screening and identifying “calprotectin suppressing drugs” (CSD's) is described below.

Identification of Calprotectin Suppressing Drugs (CSD's)

The ultimate goal and objective of screening programs for identifying compounds that have CSD activity will be to identify not just one, but ultimately two different classes of nonprotein CSD drugs. One class of such drugs will bind strongly and competitively to the zinc binding sites of calprotectin molecules that have been released by neutrophil cells, thereby occupying those zinc binding sites; this will inactivate those calprotectin molecules, by rendering them unable to sequester any additional zinc that may be available. The other class of desirable drugs will suppress the release of calprotectin molecules, by neutrophil cells that carry calprotectin. Either of those two classes of drugs can be highly useful, on its own, in helping reduce and control the localized zinc deficits that arise from excessive calprotectin levels in various types of diseases. However, some cases of the various diseases disclosed herein may respond better to one of those two classes than to the other class; and, in some instances (especially involving severe and intense cases of such diseases), both drugs, acting simultaneously, may be able to offer additive or synergistic benefits that, when combined, may be substantially more effective than either drug can achieve by itself. Accordingly, identification of drug candidates that can achieve either of those two different activities (suppression of calprotectin release by neutrophil cells, or suppression of zinc binding by already-released calprotectin molecules) should be regarded as useful and valuable goals and objectives of such screening programs.

Screening and Identification of Drugs That Bind to Zinc-binding Sites of Calprotectin

The comments in this section relate to drug candidates that can competitively bind to the zinc-binding sites of calprotectin. This goal is different and distinct from the goal of suppressing calprotectin release by neutrophil cells, which is discussed in a different section.

At the current time, no agents are known and available that have a combination of safety and efficacy traits (with tolerable side-effect profiles) that would render them suitable for administration to humans in dosages that can selectively reduce and control calprotectin activity levels, by binding to the zinc-binding sites of calprotectin molecules that have already been released by neutrophil cells. Accordingly, this invention discloses various factors and insights that can help guide the screening, identification, development, and testing of nonprotein drugs that can enable beneficial treatments of excessive calprotectin activity levels that are damaging tissues.

One set of factors that should be recognized from the outset is that: (i) it is feasible and practical, using well-known methods, to create, screen, identify, and reproduce monoclonal antibodies that will bind strongly and competitively to the zinc-binding domains of calprotectin; (ii) it is also feasible and practical to identify the “short chain variable fragments” of such monoclonal antibodies that are actively engaged in such binding activities; (iii) it is feasible and practical to generate those relatively short protein fragments in any desired quantities, either using chemical synthesis techniques, or using fermentation of suitable host cells that have been transformed with plasmids or other vectors carrying chimeric genes that encode the desired polypeptide fragments. Selected host cells also can be chosen that will carry out glycosylation and/or other post-translational processing, if desired.

Accordingly, calprotectin-binding polypeptide molecules (the terms “protein” and “polypeptide” are used interchangeably herein, to refer to any chain of amino acids that are coupled together by peptide bonds, and that have sufficient length and size to function effectively in a manner as disclosed herein) are highly useful as research reagents, and they are specifically claimed herein, as compositions of matter, because of their utility in that field. They can be manufactured and sold, as an item of commerce, to laboratories, in a manner comparable to other research reagents. Furthermore, under some circumstances, they may turn out to be useful and valuable as human therapeutic agents, in a manner comparable to injectable insulin preparations.

However, as a general rule, polypeptides are less preferred for medical use, than non-protein drugs (often referred to as “small molecule” drugs). The reasons why drugs that comprise protein chains are generally undesirable for medical or veterinary use include: (i) polypeptide drugs are likely to be digested and degraded in the gut, if ingested orally, and therefore, they usually must be injected through the skin, as exemplified by insulin; and, (ii) polypeptide drugs run a much higher risk of triggering an allergic or immune response than non-polypeptide drugs. Those problems become even more daunting and prohibitive in view of the following factors: (i) the risks of triggering allergic or immune responses are extremely difficult to predict in humans, even if extensive data are available from testing of other animal species; (ii) these problems are likely to occur not in all human users, but in some relatively small and unpredictable fraction of human users; and, (iii) because of how the allergic and immune systems function in mammals, serious and even severe and potentially lethal allergic or immune responses can arise suddenly and unpredictably, even among people who have been using such drugs for months or years without any prior problems or warning signs.

Accordingly, the potential problems and risks that can arise from when polypeptide drugs are used can be avoided by a preferred approach, which involves the screening and identification of nonprotein drug candidates that can bind directly to the zinc-binding sites of calprotectin. These types of screening programs are ideally suited for pharmaceutical companies, which have several essential assets already available and fully operational.

First: any pharmaceutical company that does original research (as distinct from companies that only manufacture and sell drugs that have become generic and publicly available, once their patents have expired) will already have “libraries” containing dozens, hundreds, or even thousands of compounds that have already been synthesized and stored, and that can readily provide samples for screening tests.

Second: any such pharmaceutical company will already have all of the machinery, supplies, and expertise that are needed to carry out sophisticated and automated screening tests, once a particular type of activity has been clearly identified as the screening criterion. In the screening program described herein, the screening criterion is simple, straightforward, and clear: the goal is to find and identify agents that can bind competitively to the zinc-binding sites of the calprotectin protein. Based on that clearly-identifiable criterion, and building on the fact that calprotectin is a protein that has been known for decades, formed from two subunits that have been fully sequenced (as reported in Odink et al 1987 and Lagasse et al 1988), an automated screening program using computerized machinery that requires very little manpower or active supervision can be set up and run, using procedures that are well-known to those who specialize in designing and running such screening efforts.

These efforts can be rendered even faster, easier, and more reliable, by using a new type of analytical machine that will soon become publicly available. This machine has not yet been publicly announced or offered for sale, and this disclosure is based on private and personal communications, arising from the Applicant's contacts with other experts who work in the field of zinc biochemistry.

In the past, it has been very difficult for anyone to distinguish between “free” zinc versus “bound” zinc. This has been especially true, since zinc binding activities and affinities of various proteins and other molecules cover a wide range. At one end of the range are proteins such as albumen that have relatively weak binding affinities; these proteins effectively act as reservoirs, or buffers, to provide readily-released supplies of zinc if and when the need arises. At the other end of the spectrum are chelating proteins such as calprotectin, which bind strongly to zinc, and which typically lead to the elimination of surplus quantities of zinc from the body, in feces or urine. Because of number, variety, and complexity of these different binding reactions, which vary over time, it has been exceptionally difficult to distinguish between free zinc versus bound zinc, in liquids, tissue samples, or other materials that are being analyzed.

However, that situation will soon change, dramatically and in ways that will make it much easier, faster, less expensive, and more convenient to carry out various tests as described herein. This new development involves the impending availability of a rapid and convenient “free zinc meter” by a company called Neurobiotex (located in Galveston, Tex.; www.neurobiotex.com). This meter is known to the Applicant through private and personal communications with Dr. Chris Frederickson, at Neurobiotex. That meter uses specialized types of chemical reagents that will undergo a conformational change that causes them to become fluorescent, if and when they bind to free zinc ions in a liquid sample; these reagents are described in articles such as Burdette et al 2001 and 2003, and Nolan et al 2004. By providing a convenient way to utilize these reagents in a metering device, this type of meter can rapidly measure free zinc levels, and express them in a logarithmic “pZn” scale, comparable to the standard pH scale for acidity. This meter will enable physicians and researchers (or even patients, when offered for at-home use, in a manner comparable to glucose monitors for diabetics) to detect and monitor changes in available zinc in liquid or other samples (such as blood, urine, tissue biopsy samples, etc.) obtained from sites that manifest or are correlated with an inflammatory or other relevant disease process.

Thus, concentrations of free zinc in various liquids can be rapidly measured, and utilized as an effective marker or indicator, both for screening programs at pharmaceutical companies, as described above, and for evaluating the efficacy of treatment regimens that are being tested or used, in animal test, human clinical trials, or medical or veterinary use. These types of free zinc meters can allow physicians, researchers, and patients to rapidly and conveniently determine: (i) the most appropriate therapeutic agent to administer, when treatment commences; (ii) the optimal dosages of a selected agent to be administered at any particular time, in view of the potentially changing severity of a particular case of a particular disease, and (ii) appropriate adjustments (in dosages, chosen agents, etc.) to maintain optimal effectiveness of a therapeutic regimen, in a specific patient, over a span of months or years.

Returning to the types of screening tests that can be used by pharmaceutical companies to evaluate hundreds or thousands of candidate compounds, to identify one or more compounds that can bind competitively to the zinc-binding sites (also called epitopes, or domains) of calprotectin, various types of relatively simple and straightforward competitive binding assays can be used, that will allow repeated use of regenerable reagents (to reduce costs). Various types of competitive binding assays are known, and the following listing of a typical sequence of steps is intended to be illustrative rather than limiting. These steps embody a so-called “cell free” approach, which can eliminate various factors (such as issues of how well a compound can permeate into a cell) that either are not relevant (since the calprotectin that is of interest herein will have already been released by a neutrophil cell), or which can be addressed in subsequent tests if deemed relevant.

Accordingly, a typical cell-free competitive binding assay can be carried out by steps such as the following:

1. A purified preparation of calprotectin molecules is created. This can be done by using either of two main approaches, and both approaches should be tested and compared against each other, to confirm their validity. One method involves obtaining a supply of calprotectin from human neutrophils or other cell types (including, for example, certain types of reported transformed cell lines that can reproduce indefinitely, in cell culture conditions, and that can be induced to secrete or release calprotectin). The other main approach involves the fermentation of E. coli, yeast, or other host cells that have been transfected by plasmids or other vectors that carry genes that will express the two calprotectin subunit polypeptides, to obtain “recombinant” calprotectin that has the same amino acid sequence as the version formed in human neutrophils. Either type of supply can use any of various known separation methods (such as, for example, electrophoresis, isoelectric focusing, or an affinity column containing monoclonal antibodies that bind to calprotectin) to purify the calprotectin molecules.

2. The purified preparation of calprotectin molecules is immobilized on solid surfaces. Most commonly, tiny beads made of certain types of inert starch or plastic are used for this type of processing, since such beads allow several major advantages, including: (i) very large aggregate surface areas; (ii) high levels of intimate contact between the bead surfaces and the molecular components of a liquid flowing through a column loaded with beads; (iii) various options for using fluidized, stirred, or other processing methods, if and when certain types of liquids that are being processed begin to pose clogging, caking, channeling, or other problems; and, (iv) various options for promoting thorough regeneration of the beads and proteins in a column, using specialized processing methods.

3. After a calprotectin preparation has been reacted with a bead preparation, the beads are washed or rinsed, to remove any non-immobilized calprotectin molecules from the bead preparation, and the bead preparation is leaded into a suitable type of column with filters at the inlet and outlet ends.

4. A “binding buffer” is passed through the column, to equilibrate the column and establish conditions of temperature, acidity, and salinity that will promote the binding of various candidate drug to the immobilized calprotectin molecules;

5. A confirmatory and calibrating binding step is carried out, using a mixture that contains a reagent such as radiolabeled zinc isotopes, to ensure that the reaction that was used to affix the calprotectin molecules to the beads did not alter the zinc binding sites or render them inaccessible. After this confirmatory step has been completed and evaluated, the temperature, acidity, and/or salinity are increased in a manner that disrupts the binding reaction between the labeled zinc and the zinc binding sites, and the labeled zinc is rinsed out of the column. This “calibration” step can also be used to determine various additional factors, such as (i) the total binding capacity of the column, under various conditions of temperature, acidity, and/or salinity (which can then be altered or controlled, if desired, to modify that binding capacity); and, (ii) the percentage of a fixed quantity of a certain zinc isotope that will bind inside the column, when no competing drug candidates have been passed through the column.

Several different isotopes of zinc are known, and each has its own type of emission, which can be measured by a corresponding type of detector. For example, the ⁶⁵Zn isotope emits gamma rays. Gamma rays pose a danger to personnel, and ⁶⁵Zn has a half-life of about 273 days; these two factors, taken together, make ⁶⁵Zn very expensive to dispose of, as a radioactive waste. Therefore, a positron-emitting isotope, ⁶³Zn, which has a half-life of only about 38 minutes, is safer and preferable for most types of research as disclosed herein.

6. A liquid that contains a candidate calprotectin-binding drug, carried by a binding buffer liquid, is passed through the column containing the immobilized calprotectin molecules. If the drug candidate has substantial affinity for the zinc-binding sites of the calprotectin, then the drug candidate will become and will remain affixed to the calprotectin, for as long as suitable binding conditions are sustained in the column;

7. A different buffer preparation containing free zinc (which can be labeled, if desired) is then passed through the column. If the zinc binding sites have been occupied by the candidate drug, smaller quantities of zinc will become bound to any remaining accessible zinc binding sites in the calprotectin molecules in the column, and larger quantities of zinc will simply pass through the column. Either or both of those zinc quantities can be measured. For example, if radiolabeled zinc is used, then the quantity of zinc that has become “stuck” inside the column can be measured; alternately, if mixtures containing unlabeled zinc are used, then the quantity of zinc that has emerged from the column, in the effluent, can be measured, by using the “free zinc meter” described above.

8. The quantity of zinc that passes through a column, after the column has received and processed a candidate drug preparation, is compared to the quantity of zinc that passed through a column when no candidate drug preparation had been passed through the column. If there is a large differential (i.e., if a substantially larger quantity of zinc passed rapidly through the column, after the column processed a candidate drug preparation), then that differential indicates that a large number of zinc binding sites, on calprotectin molecules that remain immobilized within the column, were indeed occupied by the candidate drug; this means the candidate shows good potential, and deserves closer attention and more extensive testing. By contrast, if only a small differential is observed, it will indicate that only small numbers of zinc binding sites on the calprotectin molecules were occupied by the candidate drug, and that candidate drug does not have the desired competitive binding activity that is being sought.

9. After a binding test has been completed, the column needs to be regenerated, and a final zinc binding test must be performed, to ensure that the drug did not somehow damage the column or the calprotectin molecules in ways that would not be beneficial. This type of regeneration usually is carried by increasing any or all of the temperature, acidity, and/or salinity levels in the column, using a liquid preparation that is usually called an “elution” or “wash” buffer. Increased temperature, acidity, and/or salinity will disrupt and weaken the types of non-covalent attractions and bondings that occur within such columns, thereby allowing even tightly-bound, high-affinity reagents to be removed from the column when non-physiological conditions are reached. This type of confirmatory binding test is especially important in columns that use calprotectin, since it is composed of two different subunits that bind to each other non-covalently.

The steps described above relate to one exemplary type of binding reaction that can be used for screening purposes. Other types of competitive binding assays are also known, and can be evaluated for use as disclosed herein, if desired. For example, a system widely known as the BIACORE™ system avoids the need for radioactive isotopes or other expensive chemicals, by using a method called “surface plasmon resonance spectroscopy”, which involves alterations that occur when certain types of light are shown onto, and reflected from, thin surface films. These systems, and the probes that can be used to measure biomolecule concentrations in liquids, are described in sales literature that is available from the BiaCore Company (an offshoot of Pharmacia Biosensor AB), at www.biacore.com.

Alternately or additionally, screening tests such as described above can be performed using an approach called Immobilized Metal Affinity Chromatography (IMAC). Briefly, this system would use zinc ions that are affixed (such as through a spacer chain) to a solid surface. Calprotectin molecules are preincubated with a candidate drug that may be able to bind the zinc-binding sites of the calprotectin. The calprotectin-drug mixture is then passed across the IMAC material. If the candidate drug became bound to the zinc-binding sites of the calprotectin, then the calprotectin molecules with their already-occupied binding sites will simply pass over the IMAC material, and emerge from the column or other device. By contrast, if the candidate drug did not bind to the zinc-binding sites of the calprotectin, the calprotectin molecules will bind to the IMAC material. This allows a direct and convenient measurement of whether, and to what extent, a candidate drug will bind to the zinc-binding sites of calprotectin.

In addition to those types of “cell free assays”, other types of assays can evaluate and screen the ability of candidate drugs to block zinc chelation, by calprotectin, in ways that can actually inhibit growth. One such assay can use a well-known type of yeast, called Candida albicans (e.g., Sohnle et al 1991, 2000a, and 2000b). In this type of growth inhibition bioassay, a culture of C. albicans cells in a culture media that contains no zinc is inoculated with a second culture medium that contains zinc, but that also contains calprotectin, which has been preincubated with a candidate drug that is being screened. If the candidate drug became bound to the zinc-binding sites of the calprotectin, then the calprotectin will not sequester the zinc in the supplemental media, and the zinc will be available to the C. albicans cells, which will be able to grow. However, if the candidate drug did not bind to the zinc-binding sites of the calprotectin, then the calprotectin will sequester the zinc in the supplemental media, the zinc will not be available to the C. albicans cells, and the cells will not be able to grow. C. albicans cells are highly susceptible to zinc levels, not just on a qualitative level (i.e., where the cells either grow or don't grow), but on a quantitative level, where the extent of cell growth (which can be measured by means such as glucose uptake levels, turbidity, colony sizes, etc.) is directly proportional to zinc concentrations, and provides a reliable and reproducible numerical indicator of how much available zinc remained in the supplemental culture media, after any “unoccupied” calprotectin molecules took away some portion of the zinc in the known media.

Still other types of assays might be developed in ways that are analogous to the drug discovery programs that led to zinc-displacing drugs as “ACE inhibitors”. The angiotensin converting enzyme (ACE) is crucially important in regulating blood pressure, and ACE inhibitors are widely used to control high blood pressure (hypertension). Various ACE inhibitors (including captopril, sold under the trademark CAPOTEN, and enalapril, sold under the trademark VASOTEC) were identified by their ability to displace zinc, in ACE enzyme molecules.

More recently, similar approaches were used to identify hydroxyamic acid derivatives that can displace zinc in matrix metalloproteinases (MMP's). This type of zinc displacement is of medical interest, because drugs that can suppresses the activity of MMP enzymes appear to be useful for treating some types of cancer and inflammatory diseases.

Accordingly, anyone interested in the assays disclosed herein should study the history of how those drugs were identified and tested.

Any or all of the foregoing types of screening methods can be enhanced, if one or more known types of “starting point” or “baseline” compounds are known, and can be used as benchmarks, reference points, etc. This is indeed possible, because several specific compounds are already known to bind, with at least some level of affinity and specificity, to the zinc binding sites of calprotectin.

For example, an interesting set of compounds that can offer potentially useful “baseline” compounds, in an effort to identify and develop better analogs, comprises certain types of plant alkaloids called lycorine and lycoricidinol. As described in Yui et al 1998 and 2003, and Mikami et al 1999, these compounds appear to inhibit zinc binding by calprotectin, to some extent (it should be noted that zinc binding was not measured directly, in the reports cited above; instead, those researchers measured apoptosis, which is dependent to some extent and in various ways on zinc concentrations). However, those reports also indicated that lycorine and lycoricidinol also appear to inhibit another important type of enzyme known as “tumor necrosis factor” (TNF), which plays a number of important and useful roles in mammals. Therefore, various analogs, derivatives, and other variants of those two alkaloids can and should be evaluated, in an effort to determine whether some particular variant can inhibit calprotectin without having unwanted effects on TNF proteins.

Other compounds that may offer useful starting or baseline compounds for inhibiting zinc binding by calprotectin include arachidonic acid, an omega-6 fatty acid (Kerkhoff et al 1999a and 1999b, Klempt et al 1997), certain types of omega-3 fatty acids (Belluzzi et al 2000), and analogs of short polypeptide segments that have been isolated and sequenced from monoclonal antibodies that bind tightly to the zinc-binding sites of calprotectin.

After a candidate compound has been identified that has some level of desirable activity in suppressing calprotectin binding of zinc, various known methods can be used to create an assortment of analogs, derivatives, and other variants of the “starting” molecule, which can then be tested in a subsequent round of screening. Some methods that can be used to create such analogs, derivatives, or other variants involve known and controlled chemistry steps, to create analogs, derivatives, or other variants having known structures; however, other methods that can rapidly create much greater levels of variety have been developed, which incorporate one or more steps that will add new components, moieties, or other substituents in a fashion that can be regarded as random, or semi-random. When those types of randomizing methods are used, the resulting greater variety generally implies better feedstock, with more opportunities for substantial advancements and progress, if all of the varied results can be fed into a sophisticated and automated screening program. These methods and the libraries they can create are well known, and have given names such as combinatorial libraries, spatially addressable parallel libraries, deconvolution libraries, etc., as described in various articles and patents (e.g., Lam 1997, and U.S. Pat. Nos. 5,738,996 and 5,807,683). Accordingly, these and similar approaches can be used to generate still more screening candidates for a screening program as described herein, using the “best performer” from any already-completed round of screening to provide the “starting material” for preparing a library of analogs, variants, and other offshoots that branch out from that starting point, for a subsequent round of screening tests.

In addition, it should be noted that the crystalline structures of both subunits of calprotectin are known and published (Itou et al 2001 and 2002; also see Moncrief et al 1990, Raftery et al 1996 and 1999, Loomans et al 1998, and Rety 2000 for additional information on the folding, conformation, and structure of the subunits). Therefore, various types of computer modeling and analysis can be performed, using software and methods known to those skilled in the art, to identify good small-molecule candidates that are likely to be able to bind to the zinc binding sites of calprotectin.

Screening and Identification of Drugs That Suppress Calprotectin Release By Neutrophil Cells

As mentioned above, in addition to screening for drugs that can bind to and occupy the zinc-binding sites of calprotectin, parallel screening efforts should also be undertaken to search for drug candidates that can help suppress and reduce the release of calprotectin molecules, by neutrophil cells.

Regardless of the particular steps that will be used to carry out these types of screening test, an essential starting material comprises a preparation of neutrophil cells that contain at least reasonably high levels of calprotectin molecules in their cytoplasm (i.e., the watery fluid that fills the cell).

Neutrophils that have been separated from blood from animals (such as cows or pigs) can be used, if desired; however, this can raise questions about whether the mechanisms and suppression of calprotectin by the neutrophils closely mimics and models the same processes involving human neutrophils.

Accordingly, other supplies of various types of human calprotectin-releasing cells are known and available, and additional cell lines can be created and identified by those skilled in the art, using known methods and reagents. As mentioned above, Stroncek 2004 and Russo et al 2003 describe methods for using neutrophil surface antigens and/or neutrophil “chemotactic factors” produced by bacteria, for isolating neutrophils from blood. Alternately, an immortal (cancerous) line of cells designated as HL-60 cells (derived from a human patient suffering from leukemia) which are known to release calprotectin when stimulated by phorbol esters, is described in Kerkhoff et al 1999). Alternately, certain types of blood marrow cells (including cancerous lines that are well known and widely available in research labs) can be converted into neutrophil-producing lines, by exposing them to certain types of hormones, such as “granulocyte-macrophage colony stimulating factor” (GM-CSF), which is sold by Amgen.

As yet another option, blood marrow cells that create neutrophils can be converted (transformed) into immortal (cancerous) lines by various known methods, such as by contacting them with certain types of viruses. Still more options involve creating “merged” cell lines, which will be comparable to the immortalized “hybridoma” cell lines that secrete monoclonal antibodies. Hybridoma cell lines are created by using membrane-softening agents to merge cancerous lymphoma cells (which can reproduce endlessly) with B-cell lymphocytes that generate and secrete antibodies; after these cells have been merged together, progeny cells that happened to inherit immortalizing genes from the lymphoma cells, and antibody-producing genes from the B-cells, are identified, selected, and cloned. In a similar manner, immortalized cell lines that act like neutrophils in releasing calprotectin likely can also be created, if desired, by using similar methods.

Calprotectin release by such neutrophils (or “neutrophil-like immortalized cell lines”) in cell culture conditions, can be induced by contacting neutrophil cells that contain calprotectin with any of several known agents, such as a phorbol ester known as phorbol 12-myristate 13-acetate (PMA), as described in articles such as Kerkhoff et al 1999. The phorbol ester will activate protein kinase C, a signaling protein that will then trigger a cascade that leads to the release of calprotectin.

Accordingly, these various reagents and cell types set the stage for screening assays that can be used to test candidate drug compounds, to determine whether (and how strongly) any particular candidate drug can suppress the release of calprotectin, by neutrophil cells. One such set of steps that can be used to carry out such assays can be summarized as follows:

1. A candidate drug compound is preincubated with a population of neutrophils, at suitable conditions and for a suitable period of time.

2. The cells are then contacted by PMA or some other suitable triggering agent that stimulates calprotectin release.

3. The cell culture liquid is then tested, to determine how much calprotectin protein was released into the liquid, by the neutrophil cells. This testing can be done in any of several ways. Calprotectin protein concentrations can be measured directly, using any of various known methods, such as an enzyme-linked immuno-sorbent assay (commonly known as an ELISA assay). ELISA kits that have been developed specifically for measuring calprotectin are already commercially available, from CalPro-AS (Oslo, Norway; www.phical.com). Alternately, calprotectin levels can be measured indirectly, by measuring the levels of free zinc in a liquid, using the pZn meter that will soon become available from Neurobiotex, as described above (free zinc levels can indicate calprotectin levels, since any calprotectin released by the neutrophils will sequester and reduce the levels of free zinc in a liquid being measured).

The development of such assay conditions, and the validity, reliability, and utility of any animal-derived or immortalized neutrophil (or neutrophil-like) cell line for such assays, can be facilitated by testing the candidate cell types with any of several known drugs. For example, as described in Oyama et al 1997, two drugs called amlexanox and cromolyn are known to permeate into neutrophil cells and bind to calprotectin molecules contained in the neutrophils, in a manner that impedes and suppresses the release of calprotectin by the neutrophils.

Accordingly, when the foregoing teachings are summarized or restated in language suited for patent claims, they include the following disclosures.

A method is taught for treating a disease characterized by excessive levels of calprotectin activity in localized tissue, comprising the step of reducing concentrations of active calprotectin molecules in said localized tissue, in a targeted manner that does not cause substantial alterations in zinc concentrations in a patient's stomach or intestines. This can be done by various means, such as: (1) injecting a zinc-carrying liquid into at least one artery that provides oxygenated blood to said localized tissue, in a quantity that contains sufficient zinc to occupy and inactivate zinc binding sites in calprotectin molecules in said localized tissue, thereby converting active calprotectin molecules in said localized tissue into inactivated calprotectin molecules that no longer have zinc-binding activity, or (2) removing a quantity of circulating blood from at least one artery that provides oxygenated blood to said localized tissue, in a patient being treated for a disease characterized by excessive levels of calprotectin activity, using an extra-corporeal processing device to remove at least some calprotectin molecules that have not become bound to zinc ions, from said blood, and returning at least a portion of said blood, from which at least some calprotectin molecules have been removed, to the patient being treated.

A method is also taught for treating a disease characterized by zinc deficits in localized tissue, comprising the step of administering to a patient in need of such treatment a medicament that reduces calprotectin activity in said localized tissue, wherein said medicament is administering to said patient in a “targeted” manner. The “targeted” administration can be accomplished by various means, such as (1) injection of said medicament into a body part that will cause transport of said medicament to said localized tissue, or (2) administering a medicament that has a specific binding affinity for calprotectin.

Nonprotein drugs (and medicaments containing such drugs) are also taught, for suppressing calprotectin activity and reducing zinc deficiencies in local tissue areas, wherein such nonprotein drugs have been identified by screening tests carried out on a molecular library, and the screening tests are designed to identify compounds that suppress calprotectin activity by either (1) binding to and occupying at least one zinc binding site in human calprotectin, or (2) suppressing calprotectin release by human neutrophil cells.

In addition, polypeptides that suppress calprotectin activity are taught, wherein the polypeptides are identified by screening of a molecular library to identify polypeptides that suppress calprotectin activity, either (1) by binding to and occupying at least one zinc binding site in human calprotectin, or (2) by suppressing calprotectin release by human neutrophil cells.

Nutrikine “Shuttle” Systems

In addition to all of the foregoing, it should also be recognized that certain types of compounds, referred to herein as “nutrikines”, may be able to help deliver zinc, in therapeutic concentrations, to localized or regional tissue locations that have been stressed by calprotectin-induced zinc deficiencies. As used herein, the term “nutrikines” refers to compounds that facilitate the specific delivery of a trace nutrient to a particular site (or a particular type of tissue) within the body. Nutrikines may be endogenous (e.g. originating in or produced by the body), or exogenous (e.g. originating or produced outside of the body). Compounds that are believed to have certain properties that render them apparently capable of serving (to at least some extent) as zinc-transporting nutrikines are believed to include, for example, Protein Kinase C, melatonin (Dong et al 2003, Mei et al 2002), secretin, uroguanylin, cysteine-rich intestinal peptide (CRIP), the salivary histatin proteins, a salivary protein called gustin (also called carbonic anhydrase VI), and possibly certain types of carotenoids. An example of a potential exogenous nutrikine is provided by tetracycline, which may be able to increase the delivery of zinc to the liver (and in particular, to cells in and around the bile duct, in the liver, for treating various types of cancerous, hyperproliferative, or other conditions that affect the bile duct or surrounding tissues (e.g., Dietz et al 1991).

The potential utility of secretin as a nutrikine for use in treating autism deserves particular attention, in view of an early anecdotal report of a highly positive result, followed by clinical trials that produced mainly negative results, as reviewed in articles such as Kidd 2002, Kern et al 2004, Esch et al 2004, and Sturmey 2005. It appears, from a review of the literature, that the researchers who organized and ran those clinical trials did not adequately recognize or appreciate the roles of zinc in such treatments, and failed to take steps to supplement the secretin regimen with either systemic or targeted nutritional supplements containing zinc. Accordingly, it is disclosed herein that secretin treatment, for conditions such as autism, need to be reconsidered with specific additional attention to zinc levels and supplements, and that anyone contemplating any such research should also give specific attention to the potential role of calprotectin in such treatments.

Thus, there has been shown and described new and useful methods for treating autoimmune, inflammatory and other diseases that involve excessive and unwanted calprotectin activity, and local or regional zinc deficits. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Agren, M. S., “Zinc oxide increases degradation of collagen in necrotic wound tissue,” British Journal of Dermatology 129: 221-223 (1993)

Agren, M. S., “Studies on zinc in wound healing,” Acta Dermato-Venereology, Supplement 154: 1-36 (1990)

Andersson K B, et al, “The leucocyte L1 protein: identity with the cystic fibrosis antigen and the calcium-binding MRP-8 and MRP-14 macrophage components,” Scand J Immunol., August;28(2): 241-5 (1988)

Apelqvist, J., et al, “Topical treatment of necrotic foot ulcers in diabetic patients,” Br. J. Dermatol. 123: 787-92 (1990)

Belluzzi A, et al, “Polyunsaturated fatty acids and inflammatory bowel disease,” Am J Clin Nutr. 2000 January;71 (1 Suppl): 339S-42S

Burdette S C, et al, “Fluorescent sensors for Zn(2+) based on a fluorescein platform: synthesis, properties and intracellular distribution,” J Am Chem Soc 2001 123: 7831-41

Burdette S C, et al, “ZP4, an improved neuronal Zn2+ sensor of the Zinpyr family,” J Am Chem Soc 2003 125: 1778-87

Dietz D D, et al, “Toxicity studies of acetone administered in the drinking water of rodents,” Fundam Appl Toxicol. 1991 August;17(2): 347-60

Dong W G, et al, “Effects of melatonin on the expression of iNOS and COX-2 in rat models of colitis,” World J Gastroenterol. 2003 June;9(6): 1307-11

Esch B, et al, “Secretin as a treatment for autism: a review of the evidence,” J Autism Dev Disord. 2004 October;34(5):543-56

Hallbook, T., et al, “Serum-zinc and healing of venous leg ulcers,” Lancet ii: 780-782 (1972)

Henzel J. M., et al, “Zinc concentrations within healing wounds” Archives of Surgery 100: 349-357 (1970)

Husain, S. L., “Oral zinc sulfate in leg ulcers,” Lancet 1969: 1069-71 (1969)

Isaksen B, et al, “Calprotectin inhibits matrix metalloproteinases by sequestration of zinc,” Mol Pathol. October; 54(5): 289-92 (2001)

Itou H, et al, “The crystal structure of human MRP14 (S100A9), a Ca(2+)-dependent regulator protein in inflammatory process.” J Mol Biol. 2002 Feb. 15;316(2): 265-76

Itou H, et al, “Expression, purification, crystallization and preliminary X-ray diffraction analysis of the human calcium-binding protein MRP14 (S100A9),” Acta Crystallogr D Biol Crystallogr. 2001 August;57(Pt 8): 1174-6

Kerkhoff C, “The two calcium-binding proteins, S100A8 and S100A9, are involved in the metabolism of arachidonic acid in human neutrophils,” J Biol Chem. 1999b Nov. 12;274(46): 32672-9

Kerkhoff C, et al, “Zinc binding reverses the calcium-induced arachidonic acid-binding capacity of the S100A8/A9 protein complex,” FEBS Lett. 1999a Oct. 22;460(1): 134-8

Kern J K, et al, “The effectiveness of secretin in the management of autism,” Expert Opin Pharmacother. 2004 February;5(2):379-87

Kidd P M, “Autism, an extreme challenge to integrative medicine. Part 2: medical management,” Altern Med Rev. 2002 December;7(6):472-99

Klempt M, et al, “The heterodimer of the Ca2+-binding proteins MRP8 and MRP14 binds to arachidonic acid,” FEBS Lett. 1997 May 12;408(1): 81-4

Lagasse E, et al, “Cloning and expression of two human genes encoding calcium-binding proteins that are regulated during myeloid differentiation,” Mol Cell Biol. 1988 June;8(6): 2402-10

Loomans H J, et al, “Histidine-based zinc-binding sequences and the antimicrobial activity of calprotectin,” J Infect Dis. March;177(3): 812-4 (1998)

Mei Q, et al, “Melatonin reduces colon immunological injury in rats by regulating activity of macrophages,” Acta Pharmacol Sin. 2002 October;23(10): 882-6

Mikami M, et al, “Suppressive activity of lycoricidinol (narciclasine) against cytotoxicity of neutrophil-derived calprotectin, and its suppressive effect on rat adjuvant arthritis model,” Biol Pharm Bull. 1999 July;22(7): 674-8

Moncrief N D, et al, “Evolution of EF-hand calcium-modulated proteins. I. Relationships based on amino acid sequences,” J Mol Evol., June;30(6): 522-62 (1990)

Nolan E M, et al, “The zinspy family of fluorescent zinc sensors: syntheses and spectroscopic investigations,” Inorg Chem 2004 43: 8310-7

Odink K, et al, “Two calcium-binding proteins in infiltrate macrophages of rheumatoid arthritis,” Nature, 1987 Nov. 5-11;330(6143): 80-2

Oyama Y, et al, “Two distinct anti-allergic drugs, amlexanox and cromolyn, bind to the same kinds of calcium binding proteins, except calmodulin, in bovine lung extract,” Biochem Biophys Res Commun. 1997 Nov. 17;240(2): 341-7

Pardo A, et al, “MMP-1: the elder of the family,” Int J Biochem Cell Biol. 2005 February;37(2): 283-8

Pories, Walter J., et al, “Acceleration of healing with zinc sulfate,” Annals of Surgery 165: 432-436 (1967)

Raftery M J, et al, “Overexpression, oxidative refolding, and zinc binding of recombinant forms of the murine S100 protein MRP14 (S100A9),” Protein Expr Purif. 1999 March;15(2): 228-35

Raftery M J, et al, “Isolation of the murine S100 protein MRP 14 (14 kDa migration-inhibitory-factor-related protein) from activated spleen cells: characterization of post-translational modifications and zinc binding,” Biochem J., May 15;316 (Pt 1): 285-93 (1996)

Rety S, et al, “Structural basis of the Ca(2+)-dependent association between S100C (S100A11) and its target, the N-terminal part of annexin I,” Structure Fold Des. 2000 Feb. 15;8(2): 175-84

Russo T A, et al, “Human neutrophil chemotaxis is modulated by capsule and O antigen from an extraintestinal pathogenic Escherichia coli strain,” Infect Immun. 2003 November;71(11):6435-45

Saito Y, et al, “Hyperzincemia with systemic inflammation: a heritable disorder of calprotectin metabolism with rheumatic manifestations” J Pediatr. 2002 February;140(2): 267-9

Sampson B, et al, “A case of hyperzincemia with functional zinc depletion: a new disorder?,” Pediatr Res. 1997 August;42(2): 219-25

Savlov, E. D., et al, “Radiozinc studies in experimental wound healing,” Journal of Surg. Res. 2: 209-212 (1962)

Sohnle P G, et al, “Zinc-reversible antimicrobial activity of recombinant calprotectin (migration inhibitory factor-related proteins 8 and 14),” J Infect Dis. 2000b October;182(4): 1272-5.

Sohnle P G, et al, “Effect of zinc-reversible growth-inhibitory activity in human empyema fluid on antibiotic microbicidal activity,” Antimicrob Agents Chemother January;44(1): 139-42 (2000a)

Sohnle P G, et al, “The zinc-reversible antimicrobial activity of neutrophil lysates and abscess fluid supernatants,” J Infect Dis. 1991 July;164(1): 137-42

Steinbakk M, et al, “Antimicrobial actions of calcium binding leucocyte L1 protein, calprotectin,” Lancet, September 29; 336(8718): 763-5 (1990)

Striz I, et al, “Calprotectin—a pleiotropic molecule in acute and chronic inflammation,” Physiol Res. 53(3): 245-53 (2004)

Stromberg, H. E., et al, “Topical zinc oxide treatment improves arterial and venous leg ulcers,” Br. J. Dermatol. 111: 461-8 (1984)

Stroncek D, “Neutrophil Antigens and Antibodies,” ASHI 2004 Second Quarter:54-56

Sturmey P, “Secretin is an ineffective treatment for pervasive developmental disabilities: a review of 15 double-blind randomized controlled trials,” Res Dev Disabil. 2005 January-February;26(1):87-97

Tibble J, et al, “A simple method for assessing intestinal inflammation in Crohn's disease,” Gut. October;47(4): 506-13 (2000)

Vandal K, et al, “Blockade of S100A8 and S100A9 suppresses neutrophil migration in response to lipopolysaccharide,” J Immunol. September 1;171(5): 2602-9 (2003)

Woessner J F, “Matrix metalloproteinases and their inhibitors in connective tissue remodeling,” FASEB J. 1991 May;5(8): 2145-54

Yui S, et al, “The inhibitory effect of lycorine on tumor cell apoptosis induced by polymorphonuclear leukocyte-derived calprotectin.” Immunopharmacology. 1998 August;40(2): 151-62

Yui S, et al, “Calprotectin (S100A8/S100A9), an inflammatory protein complex from neutrophils with a broad apoptosis-inducing activity,” Biol Pharm Bull. 2003 June;26(6): 753-60

Yui S, et al, “Inhibition effect of Amaryllidaceae alkaloids, lycorine and lycoricidinol on macrophage TNF-alpha production,” Yakugaku Zasshi. February; 121(2): 167-71 (2001)

1. A method for treating a disease characterized by excessive levels of calprotectin activity in localized tissue, comprising the step of reducing concentrations of active calprotectin molecules in said localized tissue, in a targeted manner that does not cause substantial alterations in zinc concentrations in a patient's stomach or intestines.

2. The method of claim 1 wherein the disease characterized by excessive levels of calprotectin activity in localized tissue is a disease characterized by both (i) chronic and localized zinc deficencies, and (ii) at least one type of autoimmune disorder.

3. The method of claim 1 wherein the disease characterized by excessive levels of calprotectin activity in localized tissue is a disease characterized by both (i) chronic and localized zinc deficencies, and (ii) at least one type of inflammatory disorder.

4. The method of claim 1 wherein the disease characterized by excessive levels of calprotectin activity in localized tissue is a disease characterized by both (i) chronic and localized zinc deficencies, and (ii) at least one type of hyperproliferative cell disorder.

5. The method of claim 1 wherein the disease characterized by excessive levels of calprotectin activity in localized tissue is selected from the group consisting of rheumatoid arthritis, cystic fibrosis, inflammatory dermatoses characterized by surplus calprotectin levels, inflammatory bowel diseases characterized by surplus calprotectin levels, and liver diseases characterized by surplus calprotectin levels.

6. The method of claim 1 wherein the disease characterized by excessive levels of calprotectin activity in localized tissue is a neurodegenerative disease.

7. The method of claim 6 wherein the neurological disease is selected from the group consisting of Alzheimer's disease, dementia, and multiple sclerosis.

8. The method of claim 1 wherein the disease characterized by excessive levels of calprotectin activity in localized tissue is a neurological disorder caused by a transitory period of calprotectin hyperactivity that led to prolonged neurological dysfunction and damage.

9. The method of claim 8 wherein the neurological disorder comprises autism.

10. The method of claim 1 wherein the disease characterized by excessive levels of calprotectin activity in localized tissue is a disease that is characterized by both (i) chronic and localized zinc deficencies, and (ii) an increase in inducible nitric oxide synthase activity.

11. The method of claim 1 wherein reduction of concentrations of active calprotectin molecules is accomplished by steps that comprise injecting a zinc-carrying liquid into at least one artery that provides oxygenated blood to said localized tissue, in a quantity that contains sufficient zinc to occupy and inactivate zinc binding sites in calprotectin molecules in said localized tissue, thereby converting active calprotectin molecules in said localized tissue into inactivated calprotectin molecules that no longer have zinc-binding activity.

12. The method of claim 1 wherein the step of reducing concentrations of calprotectin molecules that have not become bound to zinc ions, in said localized tissue, is accomplished by steps that comprise:
a. removing a quantity of circulating blood from at least one artery that provides oxygenated blood to said localized tissue, in a patient being treated for a disease characterized by excessive levels of calprotectin activity;
b. using an extra-corporeal processing device to remove at least some calprotectin molecules that have not become bound to zinc ions, from said blood; and,
c. returning at least a portion of said blood, from which at least some calprotectin molecules have been removed, to the patient being treated.

13. The method of claim 1, wherein the step of reducing concentrations of active calprotectin molecules in said localized tissue is also accompanied by administration of at least one nutrikine that promotes delivery of zinc to at least one localized tissue that previously was suffering from a zinc deficit.

14. The method of claim 13, wherein the nutrikine is selected from the group consisting of protein kinase C, melatonin, secretin, uroguanylin, cysteine-rich intestinal peptide, salivary histatin proteins, gustin, carotenoids, and tetracycline.

15. A method for treating a disease characterized by zinc deficits in localized tissue, comprising the step of administering to a patient in need of such treatment a medicament that reduces calprotectin activity in said localized tissue, wherein said medicament is administering to said patient in a targeted manner.

16. The method of claim 15 wherein targeted administration of said medicament is accomplished by injection of said medicament into a body part that will cause transport of said medicament to said localized tissue.

17. The method of claim 15 wherein targeted administration of said medicament is accomplished by administration of a medicament that has a specific binding affinity for calprotectin.

18. A nonprotein drug that suppresses calprotectin activity and reduces zinc deficiencies in local tissue areas, wherein said nonprotein drug has been identified by screening tests carried out on a molecular library, and wherein said screening tests were designed to identify compounds that suppress calprotectin activity by binding to and occupying at least one zinc binding site in human calprotectin.

19. A medicament for treating a disease characterized by excessive levels of calprotectin activity in localized tissue, comprising a nonprotein drug of claim 15, in a pharmaceutically acceptable carrier formulation.

20. A nonprotein drug that suppresses calprotectin activity and reduces zinc deficiencies in local tissue areas, wherein said nonprotein drug has been identified by screening tests carried out on a molecular library, and wherein said screening tests were designed to identify compounds that suppress calprotectin activity by suppressing calprotectin release by human neutrophil cells.

21. A medicament for treating a disease characterized by excessive levels of calprotectin activity in localized tissue, comprising a nonprotein drug of claim 17, in a pharmaceutically acceptable carrier formulation.

22. A polypeptide that suppresses calprotectin activity, wherein said polypeptide has been identified by screening tests carried out on a molecular library, and wherein said screening tests were designed to identify polypeptides that suppress calprotectin activity by binding to and occupying at least one zinc binding site in human calprotectin.

23. A polypeptide that suppresses calprotectin activity, wherein said polypeptide has been identified by screening tests carried out on a molecular library, and wherein said screening tests were designed to identify polypeptides that suppress calprotectin activity by suppressing calprotectin release by human neutrophil cells.
